(19) United States
(12) Patent Application Publication
(10) Pub. No.: US 2026/0294831 A1
(43) Pub. Date: Oct. 1, 2026

(54) PROTEIN-BASED DRUG PRODUCTS USING MONOESTER-FREE POLYSORBATE DETERGENT

(71) Applicant: NOVAVAX, INC., Gaithersburg, MD (US)

(72) Inventors: Marco CACCIUTTOLO, Gaithersburg, MD (US); Ernest MAYNARD, Gaithersburg, MD (US); Daniel SCOTT, Gaithersburg, MD (US)

(21) Appl. No.: 19/631,582

(22) Filed: Mar. 27, 2026

Related U.S. Application Data
(60) Provisional application No. 63/779,340, filed on Mar. 28, 2025.

(51) Int. Cl. A61K 9/51; A61K 39/215; A61K 39/385

(52) U.S. Cl. CPC A61K 9/5123; A61K 9/5192; A61K 39/215; A61K 39/385

(57) ABSTRACT

Compositions include a biological component and polysorbate, where the polysorbate is free of monoesters. The invention also provides pharmaceutically acceptable carriers and buffers comprising monoester-free polysorbate, detergent-core nanoparticles comprising monoester-free polysorbate, methods for producing the monoester-free polysorbate, and methods of manufacturing the pharmaceutical compositions using a monoester-free polysorbate.

### CROSS-REFERENCE TO RELATED APPLICATIONS

This application claims the benefit of priority to U.S. Provisional Application No. 63/779,340, filed on Mar. 28, 2025, the contents of which is hereby incorporated by reference herein in its entirety.

### FIELD OF THE INVENTION

The present invention generally relates to pharmaceutical compositions that contain a polysorbate with an active ingredient that comprises a biological component (e.g., protein, antigen, antibody, and the like) that can contain an esterase as a byproduct of the production and/or purification of the biological component. The polysorbate is free of monoesters. The invention also provides pharmaceutically acceptable carriers and pharmaceutically acceptable buffers comprising monoester-free (MEF) polysorbates, MEF detergent-core nanoparticles, methods for producing the monoester-free polysorbate, and methods of manufacturing the pharmaceutical composition using the monoester-free detergent.

### BACKGROUND OF THE INVENTION

Polysorbates are detergents widely used in pharmaceutical compositions such as therapeutic drug formulations, including protein formulations. Polysorbate 80 (PS80) is a mixture of partial esters of fatty acids, mainly oleic acid, with sorbitol and its anhydrides ethoxylated with approximately 20 moles of ethylene oxide for each mole of sorbitol and sorbitol anhydrides. Other polysorbates include mixtures containing different fatty acids. For example, polysorbate 60 (PS60) includes stearic acid, while polysorbate 40 (PS40) includes palmitic acid and polysorbate 20 (PS20) includes lauric acid.

It is important that all excipients in a pharmaceutical composition do not reduce the efficacy of the active pharmaceutical agent, including protein agents such as antigens used in vaccines.

### SUMMARY OF THE INVENTION

One embodiment of the invention includes a pharmaceutical nanoparticle comprising an active polypeptide ingredient associated with a polysorbate core, wherein the polysorbate is substantially free of monoesters.

In some embodiments the polysorbate comprises at least one of polysorbate 20 (PS20), polysorbate 40 (PS40), polysorbate 60 (PS60) and polysorbate 80 (PS80).

In some embodiments the polysorbate comprises PS80.

In some embodiments the active polypeptide ingredient comprises a recombinant peptide antigen.

In some embodiments a pharmaceutical composition includes the pharmaceutical nanoparticle in a pharmaceutically acceptable carrier. In some embodiments, the pharmaceutically acceptable carrier comprises a monoester-free polysorbate.

In some embodiments the pharmaceutical composition includes an adjuvant.

In some embodiments the adjuvant is a saponin adjuvant.

In some embodiments the saponin adjuvant is a Matrix adjuvant, such as Matrix-M™.

In some embodiments a method of making a pharmaceutical nanoparticle includes providing a polysorbate, separating a monoester-free fraction of the polysorbate from a monoester fraction of the polysorbate, and combining the monoester-free fraction of the polysorbate with an active polypeptide ingredient.

In some embodiments, a method of making a pharmaceutical nanoparticle, includes providing a protein extract comprising an extraction detergent; contacting the protein extract with an affinity resin; and eluting protein bound to the affinity resin with a buffer comprising a monoester-free polysorbate to obtain the pharmaceutical nanoparticle.

In some embodiments the provided polysorbate is at least one of polysorbate 20 (PS20), polysorbate 40 (PS40), polysorbate 60 (PS60), or polysorbate 80 (PS80).

In some embodiments the provided polysorbate comprises PS80.

In some embodiments separating the monoester-free fraction of the polysorbate from the monoester fraction of the polysorbate comprises a step of reverse-phase high performance liquid chromatography (HPLC).

In some embodiments, evaporative light scattering detection ELSD is used to monitor the amount of PS in the separated fractions.

In some embodiments, an average particle size of the pharmaceutical nanoparticle is measured using dynamic light scattering (DLS).

A pharmaceutically acceptable buffer or a pharmaceutically acceptable carrier comprising a monoester-free polysorbate is also provided.

In some embodiments the provided polysorbate is at least one of polysorbate 20 (PS20), polysorbate 40 (PS40), polysorbate 60 (PS60), or polysorbate 80 (PS80).

In some embodiments the provided polysorbate comprises PS80.

In some embodiments, the pharmaceutically acceptable buffer is a phosphate buffer.

### BRIEF DESCRIPTION OF THE DRAWINGS

The invention may be more completely understood in consideration of the following detailed description of various embodiments of the invention in connection with the accompanying drawings.

FIG. 1 shows the chemical structure of polysorbate 80. The total number of oxyethylene on each surfactant is w+x+y+z, and may not exceed 20 (w+x+y+z≤20).

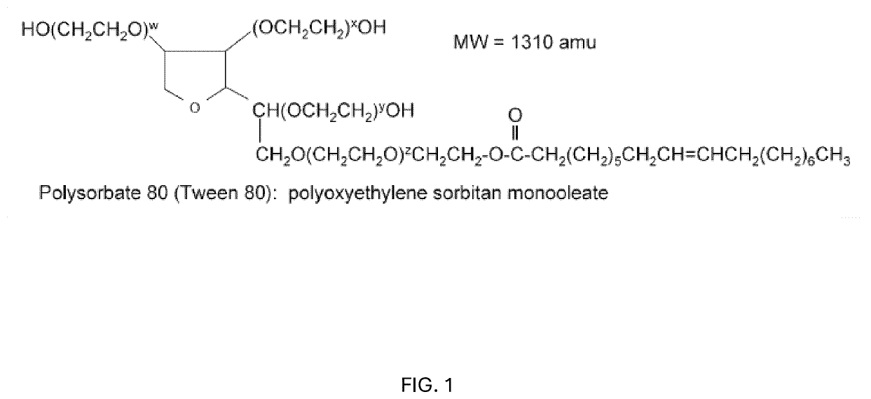

FIG. 2 shows the time-resolved measurement from reverse phase high performance liquid chromatography using an evaporative light scattering detector (RP HPLC-ELSD) where polysorbate 80 (PS80) was the input. The output includes an unesterified (PEG) fraction (eluted at about 9 minutes to about 18 minutes), a monoester fraction (eluted at about 27 minutes to about 35 minutes), and a multi-ester fraction (eluted at about 35 minutes to about 56 minutes).

FIG. 3A shows the time-resolved measurement from RP HPLC-ELSD using the monoester fraction of FIG. 2 as the input, showing that the monoester fraction was substantially separated from the other fractions of the original PS80 input.

FIG. 3B shows the time-resolved measurement from RP HPLC-ELSD using the multi-ester (monoester-free) fraction (MEF) of FIG. 2 as the input, showing that the multi-ester fraction was substantially separated from the other fractions of the original PS80 input.

FIG. 4 shows the relative potency of a Covid vaccine containing detergent core nanoparticles with viral glycoprotein over seven weeks stored at 2-8° C. for i) vaccine containing intact (unfractionated) PS80, ii) vaccine containing MEF PS80 and iii) vaccine using monoester PS80. Study controls are shown at weeks zero and seven (T0 and T7). Relative potency is a measure of antigen epitope availability in the vaccine product using an enzyme-linked immunosorbent assay and measured against a reference standard with the same molecule (recombinant SARS-COV S protein).

FIG. 5 shows time-resolved measurements from RP HPLC-ELSD of an unfractionated PS80 with a Covid antigen that was produced and purified from a cell culture. The overlayed chromatograms show changes in the monoester fraction over time, with free oleic acid becoming prominent starting at day 21. “HO Ester Region” refers to the portion of the chromatogram including the multi-ester fraction.

FIG. 6 shows time-resolved measurements from RP HPLC-ELSD of an unfractionated PS80 with a Covid antigen that was produced and purified from a cell culture. The overlayed chromatograms show a highly stable PS80 profile over at least 50 days. “HO Ester Region” refers to the portion of the chromatogram including the multi-ester fraction.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE INVENTION

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “adjuvant” refers to a compound or substance that, when used in combination with an immunogen, augments or otherwise alters or modifies the immune response induced against the immunogen. Modification of the immune response may include magnifying the response or broadening the specificity of either or both antibody and cellular immune responses. A “Matrix adjuvant” as used herein refers to a saponin adjuvant comprising a matrix component (e.g., Matrix-A, Matrix-B, Matrix-C, or the like, or any combination thereof). An example of a Matrix adjuvant is Matrix M™.

As used herein, the term “about” or “approximately” when preceding a numerical value indicates the value plus or minus a range of 10%. For example, “about 100” encompasses 90 and 110. When applied to a range, “about” indicates the lower value of the range minus 10%, and the upper value of the range plus 10%. For example, “a range of about 100 to 200” encompasses a range of 90 (lower value−10%) to 220 (upper value+10%).

As used herein, the term “biological component” refers to one or more molecule produced in a cell culture. A biological component can comprise, for example an antigen, an antibody, an enzyme, or the like. A biological component can comprise any suitable molecular type, including amino acid-based molecules (e.g., peptides, polypeptides, glycoproteins, or the like), nucleic acid-based molecules (e.g., DNA, RNA, or the like), carbohydrate-based molecules (e.g., fibers, sugars, or the like), or any combination thereof.

The terms “peptide” and “polypeptide” cover a molecule comprising two or more amino acids connected via peptide bonds. Proteins are peptides and polypeptides.

As used herein, the term “active polypeptide ingredient” refers to an active ingredient in a pharmaceutical composition that is a polypeptide. An example of an active polypeptide ingredient is a recombinant protein antigen as used in an immunogenic composition.

As used herein, the terms “immunogen,” “antigen,” and “epitope” refer to substances such as proteins, including glycoproteins and other peptides, that are capable of eliciting an immune response.

The term “substantially free of monoesters,” “monoester-free,” or “MEF” when applied to a polysorbate means that less than 5% by mass of the polysorbate is in the monoester fraction, or less than 1% or less than 0.5%.

As used herein, an “immunogenic composition” is a composition that comprises an antigen where administration of the composition to a subject results in the development in the subject of a humoral and/or a cellular immune response to the antigen.

As used herein, a “subunit” composition, for example a vaccine, that includes one or more selected antigens but not all antigens from a pathogen. Such a composition is substantially free of intact virus or the lysate of such cells or particles and is typically prepared from at least partially purified, often substantially purified immunogenic polypeptides from the pathogen. The antigens in the subunit composition disclosed herein are typically prepared recombinantly, often using a baculovirus system.

As used herein, “substantially” refers to isolation of a substance (e.g. a compound, polynucleotide, or polypeptide) such that the substance forms the majority percent of the sample in which it is contained. For example, in a sample, a substantially purified component comprises 85%, preferably 85%-90%, more preferably at least 95%-99.5%, and most preferably at least 99% of the sample. If a component is substantially replaced the amount remaining in a sample is less than or equal to about 0.5% to about 10%, preferably less than about 0.5% to about 1.0%.

The terms “treat,” “treatment,” and “treating,” as used herein, refer to an approach for obtaining beneficial or desired results, for example, clinical results. For the purposes of this disclosure, beneficial or desired results may include inhibiting or suppressing the initiation or progression of an infection or a disease; ameliorating, or reducing the development of, symptoms of an infection or disease; or a combination thereof.

“Prevention,” as used herein, is used interchangeably with “prophylaxis” and can mean complete prevention of an infection or disease, or prevention of the development of symptoms of that infection or disease; a delay in the onset of an infection or disease or its symptoms; or a decrease in the severity of a subsequently developed infection or disease or its symptoms.

As used herein an “effective dose” or “effective amount” refers to an amount of an immunogen sufficient to induce an immune response that reduces at least one symptom of pathogen infection. An effective dose or effective amount may be determined e.g., by measuring amounts of neutralizing secretory and/or serum antibodies, e.g., by plaque neutralization, complement fixation, enzyme-linked immunosorbent (ELISA), or microneutralization assay.

As used herein, the term “vaccine” refers to an immunogenic composition, such as an immunogen derived from a pathogen, which is used to induce an immune response against the pathogen that provides protective immunity (e.g., immunity that protects a subject against infection with the pathogen and/or reduces the severity of the disease or condition caused by infection with the pathogen). The protective immune response may include formation of antibodies and/or a cell-mediated response. Depending on context, the term “vaccine” may also refer to a suspension or solution of an immunogen that is administered to a subject to produce protective immunity.

As used herein, the term “subject” includes humans and other animals. Typically, the subject is a human. For example, the subject may be an adult, a teenager, a child (2 years to 14 years of age), an infant (birth to 2 year), or a neonate (up to 2 months). In particular aspects, the subject is up to 4 months old, or up to 6 months old. In some aspects, the adults are seniors about 65 years or older, or about 60 years or older. In some aspects, the subject is a pregnant woman or a woman intending to become pregnant. In other aspects, subject is not a human; for example a non-human primate; for example, a baboon, a chimpanzee, a gorilla, or a macaque, or a laboratory animal such as a mouse, rat, or rabbit. In certain aspects, the subject may be a pet, such as a dog or cat.

As used herein, the term “a pharmaceutical” means a drug or a medicine used to provide relief from a condition or to prevent a condition. Pharmaceuticals may be medicines given to cure or alleviate the symptoms of a condition or disease, and may also be vaccines or other immunological compositions administered to reduce the possibility that a disease will affect a patient or to reduce the effects of a disease if the patient is affected by the disease.

As used herein, the term “pharmaceutically acceptable” means being approved, or able to be approved, by a regulatory agency of a U.S. Federal or a state government or listed in the U.S. Pharmacopeia, European Pharmacopeia or other generally recognized pharmacopeia for use in mammals, and more particularly in humans. These compositions can be useful as a vaccine and/or antigenic compositions for inducing a protective immune response in a vertebrate.

Polysorbates, such as polysorbate 20 (PS20), polysorbate 40 (PS40), polysorbate 60 (PS60), polysorbate 65 (PS65) and polysorbate 80 (PS80) are commonly used in the formulation of parenteral drugs and vaccines. The polysorbates are also known by the trademark Tween® (Croda International, PLC) e.g. PS80 is also known as Tween® 80. PS80 is an ester between oleic acid and polyethoxylated sorbitan. Other polysorbates include esters formed using different fatty acids. For example, polysorbate 60 (PS60) includes stearic acid, while polysorbate 40 (PS40) includes palmitic acid and polysorbate 20 (PS20) includes palmitic acid.

As a surfactant, PS80 can help to maintain polypeptides in solution, and to prevent polypeptide aggregation in solution. PS80 is used, inter alia, in the formulation of “nanoparticle vaccines,” which are a mixture of the active polypeptide ingredient, such as a vaccine protein antigen, with PS80 to form a polypeptide-micelle nanoparticle. The nanoparticle structure helps to boost the immune response of the vaccine antigen.

Such nanoparticles can be prepared from a cell culture (e.g., insect cells) infected with an expression vector (e.g., a recombinant baculovirus) to express the active polypeptide ingredient of interest, such as a protein antigen, in the cell membranes. After harvesting the cells, the active polypeptide ingredient can be purified by extracting the active polypeptide ingredient from the membranes of the cells using a non-ionic extracting detergent, such as Tergitol NP-9 or Tergitol 15-S-9, both manufactured by Dow Corporation, Delaware. These detergents are not compatible with parenteral formulations to be used in humans, so the polypeptide undergoes a detergent exchange step in which the active polypeptide ingredient is bound to an affinity resin, washed with a buffer containing a polysorbate, such as PS20, PS40, PS60, PS65 or PS80, and then eluting the active polypeptide ingredient from the affinity resin in a buffer containing the polysorbate. This step leads to the formation of nanoparticles suitable for use as an active polypeptide ingredient.

Examples of vaccines formulated with active polypeptide ingredient nanoparticle drug substances include Covid, respiratory syncytial virus (RSV) and influenza vaccines. These antigens can be used as vaccines without adjuvant (i.e. unadjuvanted vaccine) or with adjuvant (i.e. adjuvanted vaccine) an adjuvant, such as Matrix M™.

### Production of Pharmaceutical Proteins

Pharmaceutical proteins, such as glycoproteins, are typically produced by recombinant expression in host cells. Standard recombinant techniques may be used. In embodiments, the proteins may be expressed in insect host cells using a baculovirus system. The cell may be transfected using any other suitable approach. In addition, other types of cell may be used, for example fungal cells. High level expression may be obtained in insect cell expression systems. Non limiting examples of insect cells include Spodoptera frugiperda (Sf) cells, e.g. Sf9, Sf21, Sf22, Trichoplusiani cells, e.g. High Five™ cells, and Drosophila S2 cells. In embodiments, the proteins described herein are produced in any suitable host cell. In embodiments, the host cell is an insect cell. In embodiments, the insect cell is an Sf9 or Sf22 cell.

Typical transfection and cell growth methods can be used to culture the cells. Vectors, e.g., vectors comprising polynucleotides that encode proteins, can be transfected into host cells according to methods well known in the art. For example, introducing nucleic acids into eukaryotic cells can be achieved by calcium phosphate co-precipitation, electroporation, microinjection, lipofection, and transfection employing polyamine transfection reagents. In one embodiment, the vector is a recombinant baculovirus.

Methods to grow host cells include, but are not limited to, batch, batch-fed, continuous and perfusion cell culture techniques. Cell culture means the growth and propagation of cells in a bioreactor (a fermentation chamber) where cells propagate and express protein (e.g. recombinant proteins) for purification and isolation. Typically, cell culture is performed under sterile, controlled temperature and atmospheric conditions in a bioreactor. A bioreactor is a chamber used to culture cells in which environmental conditions such as temperature, atmosphere, agitation and/or pH can be monitored. In one embodiment, the bioreactor is a stainless steel chamber. In another embodiment, the bioreactor is a pre-sterilized plastic bag (e.g. Cellbag®, Wave Biotech, Bridgewater, N.J.). In other embodiment, the pre-sterilized plastic bags are about 50 L to 3500 L bags.

### Purification of Pharmaceutical Proteins

After growth of the host cells, the protein may be harvested from the host cells using detergents and purification protocols. In embodiments, multiple proteins, for example viral glycoproteins, are purified simultaneously. In embodiments, host cells expressing multiple proteins are pooled together. Once the host cells have grown for 48 to 96 hours, the cells are isolated from the media and a detergent-containing solution is added to solubilize the cell membrane, releasing the protein in a detergent extract in the form of nanoparticles. Triton X-100 and TERGITOL® nonylphenol ethoxylate, also known as NP-9, are each preferred extracting detergents. The extracting detergent may be added to a final concentration of about 0.1% to about 1.0%. For example, the concentration may be about 0.1%, about 0.2%, about 0.3%, about 0.5%, about 0.7%, about 0.8%, or about 1.0%. The range may be about 0.1% to about 0.3%. In aspects, the concentration is about 0.5%.

In other aspects, different extracting detergents may be used to isolate the protein from the host cell. For example, the first detergent may be Bis(polyethylene glycol bis [imidazoylcarbonyl]), nonoxynol-9, Bis(polyethylene glycol bis [imidazoyl carbonyl]), BRIJ® Polyethylene glycol dodecyl ether 35, BRIJ® Polyethylene glycol (3) cetyl ether 56, BRIJ® alcohol ethoxylate 72, BRIJ® Polyoxyl 2 stearyl ether 76, BRIJ® polyethylene glycol monoolelyl ether 92V, BRIJ® Polyoxyethylene (10) oleyl ether 97, BRIJ® Polyethylene glycol hexadecyl ether 58P, CREMOPHOR® EL Macrogolglycerol ricinoleate, Decaethyleneglycol monododecyl ether, N-Decanoyl-N-methylglucamine, n-Decyl alpha-Dglucopyranoside, Decyl beta-D-maltopyranoside, n-Dodecanoyl-N-methylglucamide, nDodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O-(N-heptylcarbamoyl)-alpha-D-glucopyranoside, Nonaethylene glycol monododecyl ether, N-Nonanoyl-N-methylglucamine, N-NonanoyIN-methylglucamine, Octaethylene glycol monodecyl ether, Octaethylene glycolmonododecyl ether, Octaethylene glycol monohexadecyl ether, Octaethylene glycol monooctadecyl ether, Octaethylene glycol monotetradecyl ether, Octyl-beta-D glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, SPAN® 20 sorbitan laurate, SPAN® 40 sorbitan monopalmitate, SPAN® 60 sorbitan stearate, SPAN® 65 sorbitan tristearate, SPAN® 80 sorbitane monooleate, SPAN® 85 sorbitane trioleate, TERGITOL® secondary alcohol ethoxylate Type 15-S-12, TERGITOL® secondary alcohol ethoxylate Type 15-S-30, TERGITOL® secondary alcohol ethoxylate Type 15-S-5, TERGITOL® secondary alcohol ethoxylate Type 15-S-7, TERGITOL® secondary alcohol ethoxylate Type 15-S-9, TERGITOL® nonylphenol ethoxylate Type NP-10, TERGITOL® nonylphenol ethoxylate Type NP-4, TERGITOL® nonylphenol ethoxylate Type NP-40, TERGITOL® nonylphenol ethoxylate Type NP-7, TERGITOL® nonylphenol ethoxylate Type NP-9, TERGITOL® branched secondary alcohol ethoxylate Type TMN-10, TERGITOL® branched secondary alcohol ethoxylate Type TMN-6, TRITON™ X-100 Polyethylene glycol tert-octylphenyl ether or combinations thereof.

The protein, which can be associated with extraction detergent, may then be isolated from cellular debris using centrifugation. In embodiments, gradient centrifugation, such as using cesium chloride, sucrose and iodixanol, may be used. Other techniques may be used as alternatives or in addition, such as standard purification techniques including, e.g., ion exchange, affinity, and gel filtration chromatography.

For example, the first column may be an ion exchange chromatography resin, such as FRACTOGEL® EMD methacrylate based polymeric beads TMAE (EMD Millipore), the second column may be a lentil (Lens culinaris) lectin affinity resin, and the third column may be a cation exchange column such as a FRACTOGEL® EMD methacrylate based polymeric beads SO3 (EMD Millipore) resin. In other aspects, the cation exchange column may be an MMC column or a Nuvia C Prime column (Bio-Rad Laboratories, Inc). In some embodiments, the methods disclosed herein do not use a detergent extraction column (e.g., a hydrophobic interaction column) where a detergent-core nanoparticle is desired. Such a column is often used to remove detergents during purification.

### Formation of Polysorbate Detergent-Core Nanoparticles Via Detergent Exchange

To form detergent-core nanoparticles, the extracting detergent used to extract the protein from the host cell is substantially replaced with a second detergent to arrive at the nanoparticle structure. NP-9 is a preferred extraction detergent. The second detergent is typically selected from PS20, PS40, PS60, PS65, and PS80. In some embodiments, the second detergent is PS80. Typically, the detergent-core nanoparticles do not contain detectable amounts of extraction detergent (e.g., NP-9) when measured by HPLC following the detergent exchange.

In particular aspects, detergent exchange is performed using affinity chromatography to bind glycoproteins via their carbohydrate moiety. For example, the affinity chromatography may use a legume lectin column. Legume lectins are proteins originally identified in plants and found to interact specifically and reversibly with carbohydrate residues. See, for example, Sharon and Lis, “Legume lectins—a large family of homologous proteins,” FASEB J. 1990 November; 4 (14): 3198-208; Liener, “The Lectins: Properties, Functions, and Applications in Biology and Medicine,” Elsevier, 2012. Suitable lectins include concanavalin A (con A), pea lectin, sainfoin lect, and lentil lectin. Lentil lectin is a preferred column for detergent exchange due to its binding properties. Lectin columns are commercially available; for example, Capto Lentil Lectin is available from GE Healthcare. In certain aspects, the lentil lectin column may use a recombinant lectin. At the molecular level, it is thought that the carbohydrate moieties bind to the lentil lectin, freeing the amino acids of the protein to coalesce around the detergent resulting in the formation of a detergent core providing nanoparticles having multiple copies of the antigen, e.g., glycoprotein oligomers which can be dimers, trimers, or tetramers anchored in the detergent. In embodiments, the glycoproteins form trimers. In embodiments, the glycoprotein trimers are anchored in detergent. In embodiments, each glycoprotein nanoparticle contains at least one trimer associated with a non-ionic core.

The detergent, when incubated with protein (e.g., glycoprotein) to form the detergent-core nanoparticles during detergent exchange, may be present at up to about 0.1% (w/v) during early purifications steps and this amount is lowered to achieve the final nanoparticles having optimum stability. For example, the non-ionic detergent (e.g., PS80) may be about 0.005% (v/v) to about 0.1% (v/v), for example, about 0.005% (v/v), about 0.006% (v/v), about 0.007% (v/v), about 0.008% (v/v), about 0.009% (v/v), about 0.01% (v/v), about 0.015% (v/v), about 0.02% (v/v), about 0.025% (v/v), about 0.03% (v/v), about 0.035% (v/v), about 0.04% (v/v), about 0.045% (v/v), about 0.05% (v/v), about 0.055% (v/v), about 0.06% (v/v), about 0.065% (v/v), about 0.07% (v/v), about 0.075% (v/v), about 0.08% (v/v), about 0.085% (v/v), about 0.09% (v/v), about 0.095% (v/v), or about 0.1% (v/v) PS80. In embodiments, the nanoparticle contains about 0.03% to about 0.05% non-ionic detergent (e.g., PS80). In embodiments, the nanoparticle contains about 0.01% (v/v) non-ionic detergent (e.g., PS80).

In embodiments, purified proteins can be dialyzed. In embodiments, dialysis occurs after purification. In embodiments, glycoproteins can be dialyzed in a solution comprising sodium phosphate, NaCl, and non-ionic detergent (e.g., PS80). In embodiments, the dialysis solution comprising sodium phosphate contains between about 5 mM and about 100 mM of sodium phosphate, for example, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, or about 100 mM sodium phosphate. In embodiments, the pH of the solution comprising sodium phosphate is about 6.5, about 6.6, about 6.7, about 6.8, about 6.9, about 7.0, about 7.1, about 7.2, about 7.3, about 7.4, or about 7.5. In embodiments, the dialysis solution comprising sodium chloride comprises about 50 mM NaCl to about 750 mM NaCl, for example, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM, about 500 mM, about 510 mM, about 520 mM, about 530 mM, about 540 mM, about 550 mM, about 560 mM, about 570 mM, about 580 mM, about 590 mM, about 600 mM, about 610 mM, about 620 mM, about 630 mM, about 640 mM, about 650 mM, about 660 mM, about 670 mM, about 680 mM, about 690, about 700 mM, about 710 mM, about 720 mM, about 730 mM, about 740 mM, or about 750 mM NaCl. In embodiments, the dialysis solution comprising non-ionic detergent (e.g., PS80) comprises about 0.005% (v/v), about 0.006% (v/v), about 0.007% (v/v), about 0.008% (v/v), about 0.009% (v/v), about 0.01% (v/v), about 0.015% (v/v), about 0.02% (v/v), about 0.025% (v/v), about 0.03% (v/v), about 0.035% (v/v), about 0.04% (v/v), about 0.045% (v/v), about 0.05% (v/v), about 0.055% (v/v), about 0.06% (v/v), about 0.065% (v/v), about 0.07% (v/v), about 0.075% (v/v), about 0.08% (v/v), about 0.085% (v/v), about 0.09% (v/v), about 0.095% (v/v), or about 0.1% (v/v) non-ionic detergent. In some embodiments, a dialysis solution can include an MEF polysorbate (e.g., MEF PS80). In embodiments, a dialysis solution comprises about 25 mM sodium phosphate (pH 7.2), about 300 mM NaCl, and about 0.01% (v/v) PS80.

In embodiments, a nanoparticle can be stored in a pharmaceutically acceptable buffer comprising a non-ionic detergent (e.g., PS20, PS40, PS60, PS65, or PS80). In some embodiments, a buffer can comprise a MEF polysorbate. In some embodiments, a pharmaceutically acceptable buffer can comprise about 0.01% to about 1% non-ionic detergent (e.g., MEF PS80). In some embodiments, a pharmaceutically acceptable buffer can be a phosphate buffer. In some embodiments, a pharmaceutically acceptable buffer can contain a phosphate salt, a chloride salt, an amino acid, and/or a sugar. In embodiments, a buffer can comprise 10 mM sodium phosphate, 150 mM NaCl, 100 mM arginine, 5% trehalose, and 0.03% PS80 at a pH of 7.5. In embodiments, the pharmaceutically acceptable buffer comprises 25 mM sodium phosphate, 300 mM NaCl, and 0.03% PS80 at a pH of 7.2. In embodiments, the buffer comprises 25 mM sodium phosphate, 600 mM NaCl, and 0.01% PS80 at a pH of 6.8.

Detergent exchange may be performed with polypeptides purified as discussed above and purified, frozen for storage, and then thawed for detergent exchange. The resulting nanoparticle is comprised of a polysorbate detergent (e.g., MEF polysorbate) micelle that acts as a core, with hydrophobic ends of the polypeptides in the core. Thus, the nanoparticle may have a single core with multiple polypeptides attached around its surface.

The stability of compositions disclosed herein may be measured in a variety of ways. In one approach, a peptide map may be prepared to determine the integrity of the protein after various treatments designed to stress the nanoparticles by mimicking harsh storage conditions. Thus, a measure of stability is the relative abundance of peptides in a stressed sample compared to a control sample. For example, the stability of nanoparticles containing the proteins may be evaluated by exposing the nanoparticles to various pHs, proteases, salt, oxidizing agents, including but not limited to hydrogen peroxide, various temperatures, freeze/thaw cycles, and agitation. It is thought that being anchored in the detergent core provides enhanced stability for the protein by reducing undesirable interactions. For example, the improved protection against protease-based degradation may be achieved through a shielding effect whereby anchoring the proteins into the core at the molar ratios disclosed herein results in steric hindrance blocking protease access. Stability may also be measured by monitoring intact proteins, epitope availability, enzyme functionality, and/or antibody binding.

### Monoester-Free (MEF) Polysorbates

Polysorbates obtained from commercial sources, such as NOF America Corporation, San Francisco, CA or Croda, Alabaster, AL contain a mixture of monoesters and higher order esters, including di-esters, tri-esters, and tetra-esters.

Many different biological compositions (e.g., biopharmaceuticals, vaccines, and the like) include polysorbates, such as PS20, PS40, PS60, PS65, and PS80, to stabilize biological components (e.g., proteins, antigens, antibodies, and the like), reduce surface adhesion of biological components, reduce particle interaction, provide structure (e.g., by forming nanoparticles with proteins), among other functions.

Esterases are inherent to cells grown in culture, as it is part of their normal metabolism. Because biological compositions are often produced in cell cultures, they often contain small amounts of esterases. Most esterases hydrolyze monoesters of polysorbates, but are not able to hydrolyze the high order esters. The resulting product from hydrolysis of the monoester fraction are free fatty acid and polyethoxylated sorbitan. Although esterases can degrade monoesters of polysorbates, the fact that the multi-ester fraction of a polysorbate withstands attack by esterases of a lysed cell enables the polysorbate to function in biological compositions, such as by forming active polypeptide ingredient micelles, or nanoparticles.

However, it is believed that the fatty acids resulting from hydrolysis of monoesters of polysorbates interact with hydrophobic pockets in proteins, which can impact immunogenicity and/or functional attributes of such proteins, and so it is advantageous to reduce the amount of free fatty acid. For example, it has recently been discovered that the free oleic acid released from the hydrolyzation of monoesters in PS80 in Covid vaccine interacts with the recombinant protein Covid vaccine antigen. The assay used to measure Covid vaccine potency depends on the proper recognition of binding sites (epitopes) in the spike protein. If one or more epitopes are blocked (e.g. by interaction with oleic acid) the measured potency of the product is reduced.

Furthermore, even in biological compositions that are not significantly impacted by association of fatty acids with proteins contained therein, degradation of esterases can impact the stability of the polysorbate itself, which can degrade its functionality in such biological compositions. Thus, it is desirable that the hydrolyzation of polysorbate monoesters is avoided.

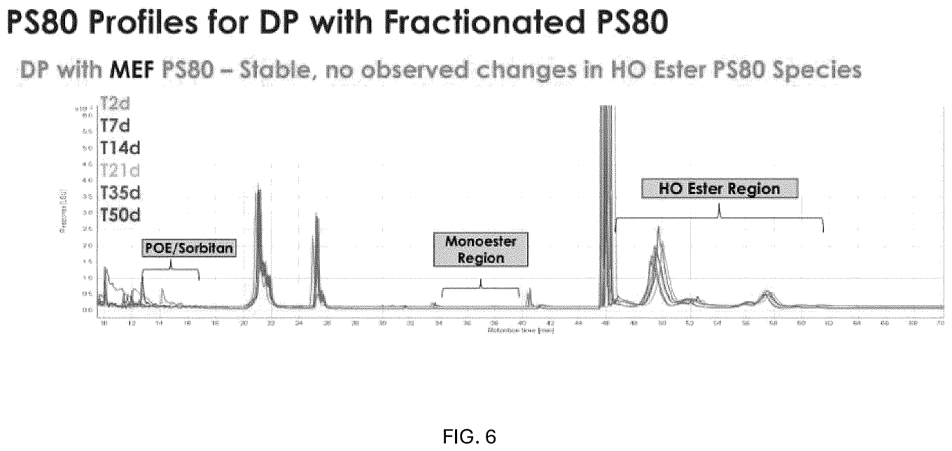

One approach to reducing the amount of free fatty acid is to avoid the use of a polysorbate that contains mono-esters, and to use a monoester-free (MEF) polysorbate that is substantially only multi-esters, e.g. di-esters, tri-esters and tetra-esters. One possible technique for separating the mono-ester fraction of a polysorbate from the multi-ester fraction is high performance liquid chromatography (HPLC). The amount of monoester remaining in a MEF polysorbate is preferably less than 5% by weight, more preferably less than 1% by weight, and even more preferably less than 0.5% by weight. Such a MEF polysorbate can be highly stable over storage with a biological component over time (see, FIG. 6).

MEF polysorbates can be used in a number of biological compositions to reduce the impact of monoester hydrolysis on such biological compositions. For example, MEF polysorbate (e.g., MEF PS80) can be used in a detergent exchange step to produce nanoparticles with a MEF polysorbate core. In another example, MEF polysorbate can be included in a pharmaceutically acceptable carrier and/or a pharmaceutically acceptable buffer.

### Adjuvants

Saponin-based adjuvants, i.e. adjuvants containing saponin, may also be used. Saponins are glycosides extracted from the bark of the Quillaja saponaria Molina tree. These bark extracts contain a heterogeneous mixture of hundreds of related saponins with structurally different glycosylation or acylation patterns that also affect their biological activities.

In some embodiments, the compositions disclosed herein may be combined with one or more adjuvants to enhance an immune response. In other embodiments, the compositions are prepared without adjuvants and are thus available to be administered as adjuvant-free compositions. Advantageously, adjuvant-free compositions disclosed herein may provide protective immune responses when administered as a single dose. Alum-free compositions that induce robust immune responses are especially useful in adults about 60 and older.

Saponins are a large family of plant-derived glycoconjugates that share a triterpene structure with a variety of glycoside side chains. Saponins have traditionally been used for making soaps based on being amphipathic. Saponins now also are used for making adjuvants based on having potent immune-stimulating properties, as taught, for example by Kensil et al., U.S. Pat. No. 5,057,540.

Saponins extracted from the bark of the South American soapbark tree Quillaja saponaria Molina contain a complex heterogeneous mixture of closely related saponins with structurally different glycosylation or acylation patterns that affect their biological activities. Quillaja saponaria Molina saponins can have a high degree of glycosyl O-acylation, a low degree of glycosyl O-acylation, or no glycosyl O-acylation in their naturally occurring forms. Saponins also can be chemically modified, for example by partial or complete deacylation or degradation.

Saponins of Quillaja saponaria Molina in particular can have potent adjuvant activity, but also can be chemically unstable, show hemolytic activity, and be associated with immediate pain at injection sites. Saponin preparations based on defined compositions of purified saponin fractions of Quillaja saponaria Molina are described, for example, by Cox et al., PCT/AU1995/000670 (WO96011711).

Incorporation of saponins of Quillaja saponaria Molina into particles comprising saponin and lipid can attenuate the chemical instability, hemolytic activity, and immediate pain when injected associated with saponins. Specific examples of particles comprising saponin and lipid include saponin based particles and saponin based antigen-presenting particles, as taught, for example, by Stertman et al., Human Vaccines & Immunotherapeutics, 2023, 19 (1): 2189885, GSK's Liposome-based Adjuvant System 01 particles, as described, for example, in Didierlaurent et al., Expert Review of Vaccines, 2017, 16 (1): 55-63, and Army Liposome Formulation Q particles, as described, for example, by Alving et al., Expert Review of Vaccines, 2020, 19 (3): 279-292.

Novavax's Matrix M™ adjuvant is a formulation that is manufactured using two extracts of saponins of Quillaja saponaria Molina, termed saponin fraction A and saponin fraction C, which are described in detail below. To make Matrix M™ adjuvant, saponin fraction A and saponin fraction C are separately mixed with cholesterol and phosphatidylcholine, in the presence of the detergent Mega-10 to form dispersions of approximately 40-50 nm-sized stable cage-like structures, designated Matrix-A and Matrix-C particles. The Mega-10 detergent is removed by diafiltration. The Matrix-A and Matrix-C particles are provided in formulations in phosphate buffer solution including 137 mM sodium chloride, 2.7 mM potassium chloride, and 9.8 mM phosphate, at pH 7.2. The Matrix M™ adjuvant is obtained by mixing the Matrix-A and Matrix-C particles at a fixed weight ratio of 85:15 of Matrix-A particles to Matrix-C particles. It should be appreciated that the A (H5N1) HA glycoprotein associates with a Matrix M™ component to make the H5-MNP, in other words the HA glycoprotein associates with a Matrix-A particle or a Matrix C particle of the Matrix M™ adjuvant. Other ratios of Matrix-A and Matrix-C particles may provide improved immune response, depending on the antigen.

Considering saponins in more detail, as noted above, saponin preparations based on defined compositions of purified saponin fractions of Quillaja saponaria Molina are described, for example, by Cox et al., PCT/AU1995/000670 (WO96011711). Initially, formulation of saponins into particles was performed with a semi-purified, non-fractionated saponin extract from the bark of Quillaja saponaria Molina, termed Quil-A. This led to several of the benefits of the formulation of saponins into such particles being recognized. Ambitions to bring the technology further towards a possible product, such as an adjuvant for use in animal and human vaccines, prompted increased purification and characterization of the Quil-A extract. The tools available for separation and characterization of saponins at the time, during the mid to late 1980s, were reversed-phase high-performance liquid chromatography, also termed RP-HPLC, and thin layer chromatography, also termed TLC. The number of peaks revealed by RP-HPLC were numerous, while TLC revealed a few major bands. Saponin raw material was subjected to semi-preparative high-performance liquid chromatography, also termed HPLC, separations. By screening separated saponin materials for adjuvant activity in mice it was found that the saponins of major interest were residing in one of the major TLC bands. Further separation of the saponins by HPLC and screening for adjuvant activity and structure forming ability resulted in the definition of sub-groups of saponin fractions with different and interesting features. The major findings are summarized in TABLE 1, in which present terminology and major components are included for clarity and reference is made to what currently are termed fraction A, fraction B, and fraction C of Quillaja saponaria Molina saponins.

TABLE 1
Initial characterization of potentially useful fractions
of saponin materials obtained from Quil-A.

| | Adjuvant | | | | |
|---|---|---|---|---|---|
| | activity | Matrix | | | |
| | (non- | structure | | Mw of | Present |
| TLC | formulated | forming | Hemolytic | major known | termi- |
| band | saponin) | ability | activity | component(s) | nology |
| B1 | Poor | Poor | Low | NA | NA |
| B2 | Potent | Good | Medium | 1988 | Fraction C |
| B3 | Potent | Atypical | High | 2150 (2175 | Fraction B |
| | | structures | | and 2019) | |
| B4 | Poor/none | Good | Low | 1862 | Fraction A |

Over the years the process for fractionation of saponin raw material into fraction A and fraction C has been developed and scaled-up. For example, as described in Cox et al., PCT/AU1995/000670 (WO96011711), fractions A, B, and C can be prepared from the lipophilic fraction obtained on chromatographic separation of the crude aqueous Quillaja Saponaria Molina extract on a SEP-PAK column and elution with 70% acetonitrile in water to recover the lipophilic fraction. This lipophilic fraction can then be separated by semipreparative HPLC with elution using a gradient of from 25% to 60% acetonitrile in acidic water. Fraction A is the fraction that is eluted at approximately 39% acetonitrile. Fraction C is the fraction that is eluted at approximately 49% acetonitrile.

As noted above, saponin preparations based on defined compositions of purified saponin fractions of Quillaja saponaria Molina are described, for example, by Cox et al., PCT/AU1995/000670 (WO96011711).

In specific embodiments, Saponin Fraction C has a purity of at least 80%, as determined by HPLC. In specific embodiments, a defined ratio of specific subcomponents within Fraction A or C are identified by mass spectrometry. In specific embodiments, Cholesterol is derived from a plant-based source (phytosterol). In specific embodiments, Phosphatidylcholine comprises POPC fatty acid chains. In specific embodiments, Mega-10 detergent is used at a concentration of 1% (w/v) during particle formation.

In specific embodiments, Matrix-A and Matrix-C particles have a mean diameter between 30 nm and 50 nm, as measured by dynamic light scattering (DLS). In specific embodiments, Matrix-A and Matrix-C particles have a polydispersity index (PDI) less than 0.2. In specific embodiments, Matrix-A and Matrix-C particles exhibit a zeta potential between −20 mV and −40 mV. In specific embodiments, the cage-like structure is confirmed by transmission electron microscopy (TEM).

In specific embodiments, a Matrix-A to Matrix-C weight ratio is between 80:20 and 90:10. In some embodiments the Matrix-A to Matrix-C ratio is around 85:15. In specific embodiments, diafiltration uses a membrane with a molecular weight cut-off (MWCO) of 10 kDa to remove Mega-10. In specific embodiments, diafiltration is performed for a minimum of 10 cycles. In specific embodiments, a phosphate buffer solution has a phosphate concentration between 5 mM and 15 mM, pH 7.2. In specific embodiments, a temperature between 20° C. and 25° C. is maintained during the mixing and diafiltration steps. In specific embodiments, the Matrix M™ adjuvant is sterilized by sterile filtration using a 0.22 μm filter.

In specific embodiments, a lyoprotectant (sucrose) is included at a concentration between 5% and 10% (w/v). In specific embodiments, the final formulation pH is adjusted to between 6.8 and 7.4. In specific embodiments, the formulation is stable for at least 12 months when stored at 2-8° C. In specific embodiments, reconstitution is with sterile water.

In specific embodiments, Matrix M™ adjuvant is used with a protein antigen, such as an oncoprotein antigen. In specific embodiments, a vaccine composition comprises Matrix M™ adjuvant at a concentration between 10 μg/mL and 50 μg/mL. In specific embodiments, administration is via intramuscular (IM) or intranasal (IN) route. In specific embodiments, induction of a specific antibody isotype response (IgG1). In specific embodiments, protective efficacy is demonstrated against a specific pathogen challenge in a mouse model. In specific embodiments, the adjuvant activity displays an increase in antibody titer compared to the antigen alone. In specific embodiments, the formulation exhibits reduced reactogenicity compared to other saponin-based adjuvants.

### Pharmaceutical Formulations and Administration

Pharmaceutical compositions that include an active polypeptide ingredient and a substantially monoester-free polysorbate may be formulated with any suitable pharmaceutically acceptable ingredient for administration to a patient. For example, they may include a saline or phosphate buffer or the like.

Pharmaceutical compositions that include an active polypeptide ingredient and a substantially monoester-free polysorbate may be administered to a patient in any acceptable dosage form. In embodiments, administration is via injection, for example intramuscular, intravenous and the like.

### EXAMPLES

### Example 1: Separation of Mono-Ester and Multi-Ester Fractions of PS80

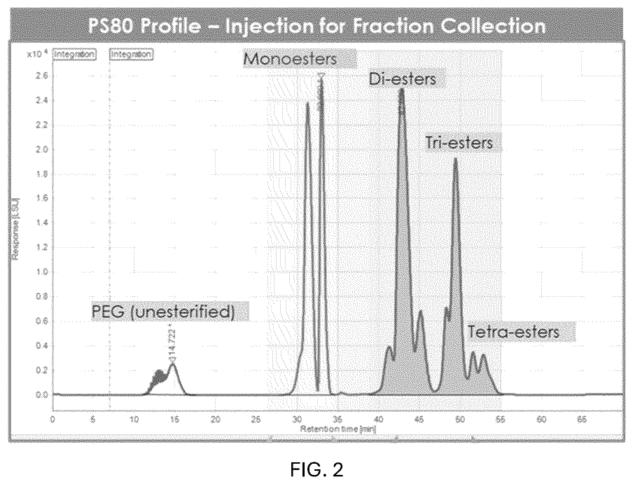

A 1% (w/v) PS80, obtained from NOF America Corporation, San Francisco, CA, was injected into a high performance liquid chromatography (HPLC) system having evaporative light scattering detection (ELSD). The output obtained using ELSD as a function of time is shown in FIG. 2. Output fractions from the HPLC system were collected based on established retention times of ester groups: polyethylene glycol (PEG, unesterified)=9-18 minutes, monoester fraction=27-35 minutes and multi-ester fraction (di-, tri-, and tetra-esters)=35-56 minutes.

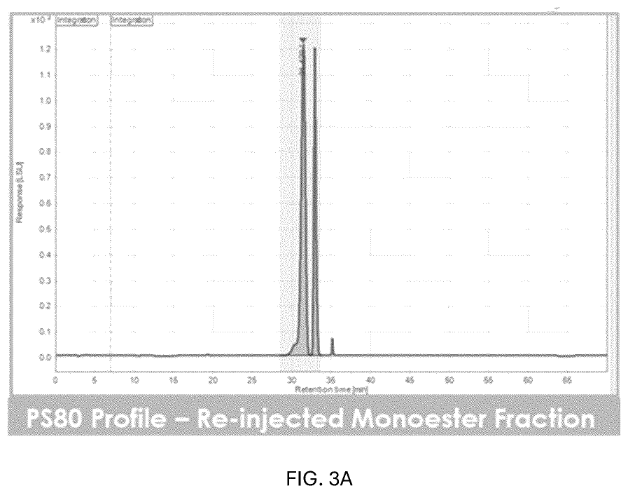

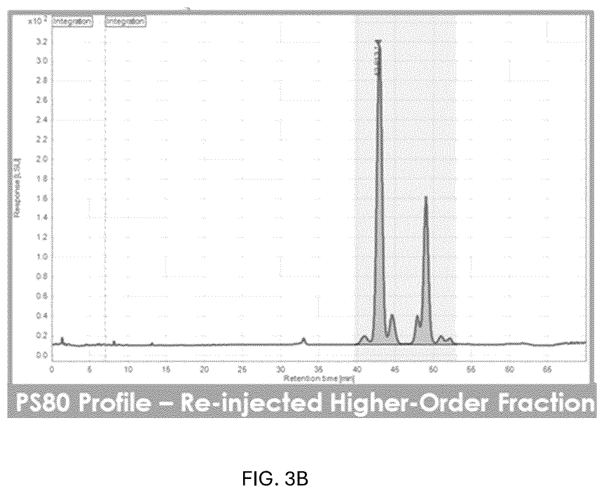

To confirm that the collected fractions were separated to an acceptable degree, the monoester fraction and the multi-ester fraction were each subsequently and separately passed through the HPLC system without any separation at the output. The test of the monoester fraction produced an ELSD output shown in FIG. 3A, showing substantial isolation of the monoester fraction from the unesterified fraction and the multi-ester fraction. The test of the multi-ester fraction produced an ELSD output shown in FIG. 3B, showing that the multi-ester fraction contained a negligible amount of the monoester fraction.

Particle sizes of the monoester-free (MEF) PS80 micelles and intact PS80 micelles were measured using dynamic light scattering. The MEF PS80 micelles had an average diameter of 30 nm, compared to the average particle diameter of the intact PS80 micelles of 15 nm.

### Example 2: Application of MEF PS80 in Drug Product-Stability Testing

To test the stability of a drug product formulated with MEF PS80, a drug product was produced in three different lots, respectively using i) intact commercial PS80, ii) MEF PS80 and iii) monoester fraction PS80. The drug product was a Covid vaccine using recombinant viral glycoprotein as the antigen, in PS80 detergent-core nanoparticles.

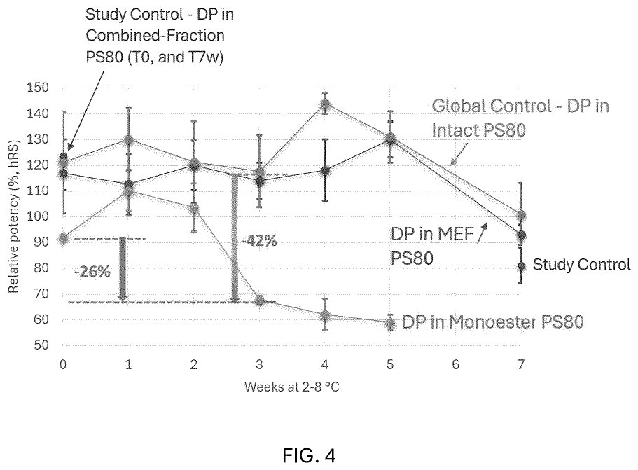

The relative potency of the drug product was measured at the start (T0) and each week over a period of seven weeks (T1-T7). Over this time the drug products were stored at a temperature of 2-8° C. The results are presented in FIG. 4A for the drug product containing intact commercial PS80 (curve 402), MEF PS80 (curve 404) monoester fraction PS80 (curve 406).

The drug product containing the monoester fraction PS80 demonstrated poor stability, losing potency after three weeks, and finishing below 60% at week 5. The drug product containing MEF PS80 demonstrated similar potency to the drug product containing the intact PS80.

The number of valid replicates over the same seven-week time period is shown in FIG. 4B.

The use of MEF polysorbates in the production of polypeptide drug product may result in a formulation that is resistant to monoester hydrolysis catalyzed by the host cell enzymes. Since the MEF polysorbate does not undergo enzymatic hydrolysis, there is no free fatty acid to interact with the active polypeptide ingredient. Aggregation number and critical micelle concentration in MEF polysorbate compositions are expected to remain stable. Use of MEF polysorbate compositions reduces the variability in drug substance and drug product attributes that could be caused by differences in the amount of esterase impurity present.

The use of MEF polysorbate eliminates complication from monoester hydrolysis and fatty acid formation, ensuring that important detergent parameters such as the critical micelle concentration (CMC) value will remain constant during the time of test and be comparable between sample and reference material, if used for generating a reportable result.

MEF polysorbates are expected to have a lower CMC value since they have a higher degree of esterification compared to intact polysorbate, where a significant proportion of the detergent exists as monoester forms. Lower CMC values advantageously require lower amounts of excipient to achieve micelle formation and detergent function.

### Example 3: MEF PS80 Stability Testing

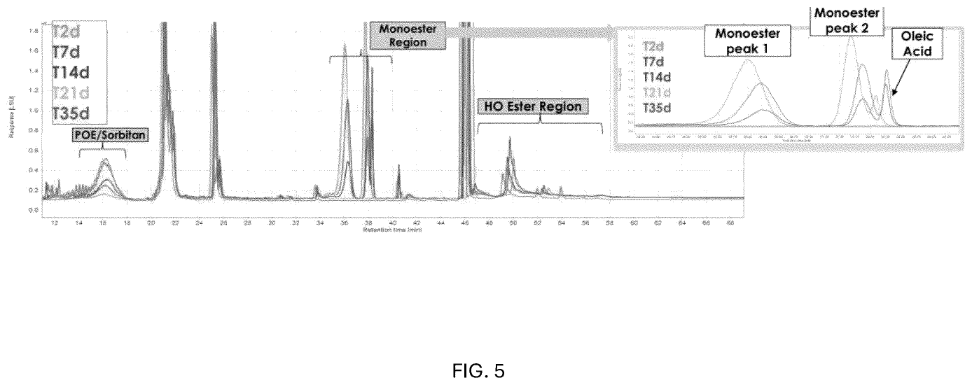

To test the stability of MEF PS80, drug product (DP) compositions were made from a Covid antigen that was produced and purified from a cell culture, and a buffer comprising either unfractionated PS80 or MEF PS80. The DP compositions were stored at 2-8° C. for up to 50 days and the PS80 from each composition was subjected to RP HPLC-ELSD to assess the stability of the PS80 over storage. As can be seen in FIG. 5, the monoester region of the overlayed chromatograms show that the monoesters were unstable in unfractionated PS80, with free oleic acid appearing by day 21 (see, the insert of FIG. 5). In contrast, FIG. 6 shows that no apparent free oleic acid was formed in MEF PS80, and the multi-ester region was surprisingly stable over at least 50 days of storage.

### ENUMERATED EMBODIMENTS

1. A pharmaceutical nanoparticle comprising an active polypeptide ingredient associated with a polysorbate core, wherein the polysorbate is substantially free of monoesters.
2. The pharmaceutical nanoparticle of Embodiment 1, wherein the polysorbate comprises at least one of polysorbate 20 (PS20), polysorbate 40 (PS40), polysorbate 60 (PS60) and polysorbate 80 (PS80).
3. The pharmaceutical nanoparticle of Embodiment 2, wherein the polysorbate comprises PS80.
4. A pharmaceutical composition comprising the pharmaceutical nanoparticle of Embodiment 1 in a pharmaceutically acceptable carrier.
5. The pharmaceutical composition of Embodiment 4, further comprising an adjuvant.
6. The pharmaceutical composition of Embodiment 5, wherein the adjuvant is a saponin adjuvant, such as a Matrix adjuvant.
7. A method of making a pharmaceutical nanoparticle, comprising:
   i) providing a polysorbate;
   ii) separating a monoester-free fraction of the polysorbate from a monoester fraction of the polysorbate; and
   iii) combining the monoester-free fraction of the polysorbate with an active polypeptide ingredient.
8. The method of Embodiment 7, wherein the polysorbate comprises at least one of polysorbate 20 (PS20), polysorbate 40 (PS40), polysorbate 60 (PS60), or polysorbate 80 (PS80).
9. The method of Embodiment 8, wherein the polysorbate comprises PS80.
10. The method of Embodiment 7, wherein separating the monoester-free fraction of the polysorbate from the monoester fraction of the polysorbate comprises a step of reverse-phase high performance liquid chromatography (RP-HPLC).
11. The method of Embodiment 10, further comprising a step of evaporative light scattering detection (ELSD) to detect the separated fractions.
12. The method of Embodiment 7, further comprising characterizing an average particle size of the pharmaceutical nanoparticle and/or polysorbate micelle using dynamic light scattering (DLS).
13. A method of making a pharmaceutical nanoparticle, comprising:
   i) providing a protein extract comprising an extraction detergent;
   ii) contacting the protein extract with an affinity resin; and
   iii) eluting protein bound to the affinity resin with a buffer comprising a monoester-free polysorbate to obtain the pharmaceutical nanoparticle.
14. The method of Embodiment 13, wherein the polysorbate comprises at least one of polysorbate 20 (PS20), polysorbate 40 (PS40), polysorbate 60 (PS60), or polysorbate 80 (PS80).
15. The method of Embodiment 14, wherein the polysorbate comprises PS80.
16. The method of Embodiment 13, further comprising characterizing an average particle size of the pharmaceutical nanoparticle and/or polysorbate micelle using dynamic light scattering (DLS).
17. A pharmaceutically acceptable buffer comprising a monoester-free polysorbate.
18. The pharmaceutically acceptable buffer of Embodiment 17, wherein the polysorbate comprises at least one of polysorbate 20 (PS20), polysorbate 40 (PS40), polysorbate 60 (PS60), or polysorbate 80 (PS80).
19. The pharmaceutically acceptable buffer of Embodiment 18, wherein the polysorbate comprises PS80.
20. The pharmaceutically acceptable buffer of Embodiment 17, wherein the buffer is a phosphate buffer.
21. A composition, comprising biological component and a monoester-free polysorbate.
22. The composition of Embodiment 21, wherein the polysorbate comprises at least one of polysorbate 20 (PS20), polysorbate 40 (PS40), polysorbate 60 (PS60), or polysorbate 80 (PS80).
23. The composition of Embodiment 21, wherein the polysorbate comprises PS80.
24. The composition of Embodiment 21, wherein the composition is a pharmaceutical composition.
25. The composition of Embodiment 24, wherein the monoester-free polysorbate is a component of a pharmaceutically acceptable buffer.
26. The composition of Embodiment 24, wherein the biological component is an active polypeptide ingredient.
27. The composition of Embodiment 26, wherein the active polypeptide ingredient comprises an antigen or an antibody.
28. The composition of Embodiment 26, further comprising an adjuvant.
29. The composition of Embodiment 28, wherein the adjuvant is a saponin adjuvant, such as a Matrix adjuvant.
30. The composition of Embodiment 28, wherein the composition is a vaccine.

Various modifications, equivalent processes, as well as numerous compositions to which the present invention may be applicable will be readily apparent to those of skill in the art to which the present invention is directed upon review of the present specification. The claims are intended to cover such modifications and compositions.

As noted above, the present invention is applicable to protein-based active pharmaceutical ingredients, and pharmaceutical compositions including such compositions. Accordingly, the present invention should not be considered limited to the particular examples described above, but rather should be understood to cover all aspects of the invention as fairly set out in the attached claims.

1. A pharmaceutical nanoparticle comprising an active polypeptide ingredient associated with a polysorbate core, wherein the polysorbate is substantially free of monoesters.

2. The pharmaceutical nanoparticle of claim 1, wherein the polysorbate comprises at least one of polysorbate 20 (PS20), polysorbate 40 (PS40), polysorbate 60 (PS60), or polysorbate 80 (PS80).

3. A pharmaceutical composition comprising the pharmaceutical nanoparticle of claim 1 in a pharmaceutically acceptable carrier.

4. The pharmaceutical composition of claim 3, further comprising an adjuvant.

5. A method of making a pharmaceutical nanoparticle, comprising:
i) providing a polysorbate;
ii) separating a monoester-free fraction of the polysorbate from a monoester fraction of the polysorbate; and
iii) combining the monoester-free fraction of the polysorbate with an active polypeptide ingredient.

6. The method of claim 5, wherein the polysorbate comprises at least one of polysorbate 20 (PS20), polysorbate 40 (PS40), polysorbate 60 (PS60), or polysorbate 80 (PS80).

7. The method of claim 5, wherein separating the monoester-free fraction of the polysorbate from the monoester fraction of the polysorbate comprises a step of reverse-phase high performance liquid chromatography (RP-HPLC).

8. The method of claim 7, further comprising a step of evaporative light scattering detection (ELSD) to detect the separated fractions.

9. The method of claim 7, further comprising characterizing an average particle size of the pharmaceutical nanoparticle using dynamic light scattering (DLS).

10. A method of making a pharmaceutical nanoparticle, comprising:
i) providing a protein extract comprising an extraction detergent;
ii) contacting the protein extract with an affinity resin; and
iii) eluting protein bound to the affinity resin with a buffer comprising a monoester-free polysorbate to obtain the pharmaceutical nanoparticle.

11. The method of claim 10, wherein the polysorbate comprises at least one of polysorbate 20 (PS20), polysorbate 40 (PS40), polysorbate 60 (PS60), or polysorbate 80 (PS80).

12. A pharmaceutically acceptable buffer comprising a monoester-free polysorbate.

13. The pharmaceutically acceptable buffer of claim 12, wherein the polysorbate comprises at least one of polysorbate 20 (PS20), polysorbate 40 (PS40), polysorbate 60 (PS60), or polysorbate 80 (PS80).

14. The pharmaceutically acceptable buffer of claim 12, wherein the buffer is a phosphate buffer.

15. A composition, comprising a biological component and a monoester-free polysorbate.

16. The composition of claim 15, wherein the polysorbate comprises at least one of polysorbate 20 (PS20), polysorbate 40 (PS40), polysorbate 60 (PS60), or polysorbate 80 (PS80).

17. The composition of claim 15, wherein the composition is a pharmaceutical composition.

18. The composition of claim 17, wherein the monoester-free polysorbate is a component of a pharmaceutically acceptable buffer.

19. The composition of claim 17, wherein the biological component is an active polypeptide ingredient.

20. The composition of claim 19, wherein the composition is a vaccine.
